SEQUENCE LISTING (20 sequences)

SEQ ID NO: 1 - protein, 7 aa, synthetic construct
GGTFSSY

SEQ ID NO: 2 - protein, 5 aa, synthetic construct
IPIFG

SEQ ID NO: 3 - protein, 13 aa, synthetic construct
EDILMIYSYFGMD

SEQ ID NO: 4 - protein, 7 aa, synthetic construct
GFNIKDT

SEQ ID NO: 5 - protein, 6 aa, synthetic construct
YPTNGY

SEQ ID NO: 6 - protein, 7 aa, synthetic construct
SRWGGDG

SEQ ID NO: 7 - protein, 142 aa, synthetic construct
MDWTWRFLFVVAAATGVQSQVQLVQSGAEVKKPGSSVKVSCKASGGTFSSYAISWVRQAPGQGLEWMGGIIPIFGTANYAQKFQGRVTITADESTSTAYMELTGLTSQDTAVYYCAREDILMIYSYFGMDVWGQGTTVTVSS

SEQ ID NO: 8 - DNA, 427 bp, synthetic construct
  source 1..427: mol_type = other DNA
atggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgaccggcctgacatctcaagacacggccgtatactactgcgcgagagaagacatacttatgatttattcgtacttcggcatggacgtctggggccaagggaccacggtcaccgtctcctcag

SEQ ID NO: 9 - protein, 136 aa, synthetic construct
MDWTWRILFLVAAATGAHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVT

SEQ ID NO: 10 - DNA, 407 bp, synthetic construct
  source 1..407: mol_type = other DNA
atggactggacctggaggatcctcttcttggtggcggccgccacaggcgcgcactccgaggtgcagctggtggagtctggtggtggtcttgttcaacctggtggttctcttcgtctttcttgtgctgcttctggttttaatattaaagatacttatattcattgggttcgtcaagctcctggtaaaggtcttgaatgggttgctcgtatttatcctactaatggttatactcgttatgctgattctgttaaaggtcgttttactatttctgctgatacttctaaaaatactgcttatcttcaaatgaactctcttcgtgctgaagatactgctgtttattattgttctcgttggggtggtgatggtttttatgctatggattattggggtcaaggtactcttgtcac

SEQ ID NO: 11 - protein, 15 aa, synthetic construct
CARGRSSSSYYMDVW

SEQ ID NO: 12 - protein, 20 aa, synthetic construct
CARASVPAPITGTIIWFDPW

SEQ ID NO: 13 - protein, 19 aa, synthetic construct
CARVESYPSEYYYFGMDVW

SEQ ID NO: 14 - protein, 14 aa, synthetic construct
CATTYSSSSGFDCW

SEQ ID NO: 15 - protein, 20 aa, synthetic construct
CARASVPAPITGTIIWFDPW

SEQ ID NO: 16 - protein, 15 aa, synthetic construct
CARGRSSSSYYMDVW

SEQ ID NO: 17 - protein, 8 aa, synthetic construct
CTHDWRNW

SEQ ID NO: 18 - protein, 20 aa, synthetic construct
CVKSAGRTSASPSYYYMDVW

SEQ ID NO: 19 - protein, 20 aa, synthetic construct
CARGRGRDCRSSNCYYMDVW

SEQ ID NO: 20 - protein, 15 aa, synthetic construct
CARGRSSSSYYMDVW

(19) United States
(12) Patent Application Publication
(10) Pub. No.: US 2025/0230260 A1
(43) Pub. Date: Jul. 17, 2025

(54) CDRS FOR GLYCOSYLATED ACAT1

(71) Applicant: H. LEE MOFFITT CANCER CENTER AND RESEARCH INSTITUTE, INC., Tampa, FL (US)

(72) Inventors: Jose CONEJO-GARCIA, Durham, NC (US); Subir BISWAS, West Bengal (IN); Gunjan MANDAL, West Bengal (IN)

(21) Appl. No.: 18/855,691

(22) PCT Filed: Apr. 17, 2023
(86) PCT No.: PCT/US2023/065840

§ 371 Date: Oct. 10, 2024

Related U.S. Application Data
(60) Provisional application No. 63/331,661, filed on Apr. 15, 2022.

(51) Int. Cl. C07K 16/40; A61K 39/00; A61P 35/00

(52) U.S. Cl. CPC C07K 16/40; A61P 35/00; C12Y 203/01009; A61K 2039/505; C07K 2317/565

(57) ABSTRACT

Disclosed are compositions and methods related to anti-ACAT1 binding molecules that preferentially bind glycosylated ACAT1.

### CROSS-REFERENCE TO RELATED APPLICATIONS

This Application claims the benefit of U.S. Provisional Application No. 63/331,661, filed on Apr. 15, 2022, which is incorporated herein by reference in its entirety.

This invention was made with government support under grant/contract numbers U01CA232758 and IK99CA266947-01 awarded by the National Institutes of Health. The government has certain rights in the invention.

### REFERENCE TO SEQUENCE LISTING

The sequence listing submitted on Apr. 17, 2023, as an .XML file entitled “10110-367WO1” created on Apr. 17, 2023, and having a file size of 19,512 bytes is hereby incorporated by reference pursuant to 37 C.F.R. § 1.52(e)(5).

### I. BACKGROUND

Immuno-oncology today is sharply focused on T cells. However, B cells can present antigens and provide co-stimulation to T cells and coordinated T and B cell activation needs to occur to maximize immune protection. In the tumor microenvironment, T and B cells frequently interact, but cooperation between humoral and cellular responses remains incompletely understood. T follicular helper (Tfh) cell-driven B cell responses play a role in the effectiveness of checkpoint inhibitors. However, the drivers of Tfh conversion at tumor beds remain unknown. Some T:B cell intra-tumoral interactions take place in highly organized conglomerates that recapitulate the architecture of secondary lymph nodes, termed tertiary lymphoid structures (TLS). Similar to TLS in autoimmune conditions, some intra-tumoral TLS contain germinal centers with interdigitating follicular dendritic cells, plus an adjacent T cell zone composed by CD4⁺ and CD8⁺ lymphocytes and high endothelial venules. The presence of TLS is associated with positive outcomes in multiple human malignancies, including ovarian, pancreatic, colorectal, bladder and non-small cell lung cancer. Given the importance of B cells to a positive outcome and the association of the presence of TLS with positive outcomes, what are needed are new binding molecules that target tumors and facilitate TLS formation.

### II. SUMMARY

Disclosed are methods and compositions related to binding molecules of ACAT1.

In one aspect, disclosed herein are anti-anti-acetyl-CoA acetyltransferase 1 (ACAT1) binding molecules (such as, for example, antibodies, diabodies, nanobodies, scFv, sFv, and immunotoxins) that specifically binds glycosylated ACAT1, and wherein the anti-ACAT1 binding molecule comprising one or more variable heavy chain complementarity determining regions (CDRs) as set forth in SEQ ID Nos 1-3 (such as, for example the variable heavy chain as set forth in SEQ ID NO: 7) and one or more variable light chain complementarity determining regions (CDRs) as set forth in SEQ ID Nos 4-6 (such as, for example the variable light chain as set forth in SEQ ID NO: 9). In one aspect, the anti-ACAT1 binding molecule is an IgG, IgA, or IgM antibody.

Also disclosed herein are methods of treating, reducing, decreasing, inhibiting, ameliorating, and or preventing a cancer and/or metastasis (such as for example, ovarian, pancreatic, colorectal, bladder and non-small cell lung cancer) comprising administering to the subject the ACAT1 binding molecule of any preceding aspect. For example, disclosed herein are methods of treating, reducing, decreasing, inhibiting, ameliorating, and or preventing a cancer and/or metastasis (such as for example, ovarian cancer) in a subject in need thereof comprising administering to the subject an anti-acetyl-CoA acetyltransferase 1 (ACAT1) binding molecule (such as, for example, antibodies, diabodies, nanobodies, scFv, sFv, and immunotoxins) that specifically binds glycosylated ACAT1, and wherein the anti-ACAT1 binding molecule comprising one or more variable heavy chain complementarity determining regions (CDRs) as set forth in SEQ ID Nos 1-3 (such as, for example the variable heavy chain as set forth in SEQ ID NO: 7) and one or more variable light chain complementarity determining regions (CDRs) as set forth in SEQ ID Nos 4-6 (such as, for example the variable light chain as set forth in SEQ ID NO: 9). In one aspect, the anti-ACAT1 binding molecule is an IgG, IgA, or IgM antibody.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, and 1E show the density of B and T cell infiltrates in ovarian cancer tumor lysis syndrome (TLS). FIG. 1A shows immunofluorescent staining for CD3, CD19, and DAPI on ovarian tumors revealing the formation of Tumor Lysis Syndrome (TLS). FIG. 1B shows a nearest neighbor analysis showing the median distance between T cells and B cells. FIG. 1C shows the median survival of TLS positive patients versus TLS negative patients. FIG. 1D shows the average number of B cells, CD8 T cells, and CD4 T cells in TLS positive and TLS negative patients. FIG. 1E shows a CIRCOS plot showing the interaction strength of B cells (CD20 positive), CD8 T cells, and CD4 T cells in TLS positive and TLS negative patients.

FIG. 2 shows highly oligoclonal antibody responses in ovarian cancer and variable heavy chain CDR3 sequences for antibodies in TLS samples. Shown are

(SEQ ID NO: 11)
CARGRSSSSYYMDVW,
(SEQ ID NO: 12)
CARASVPAPITGTIIWFDPW,
(SEQ ID NO: 13)
CARVESYPSEYYYFGMDVW,
(SEQ ID NO: 14)
CATTYSSSSGFDCW,
(SEQ ID NO: 15)
CARASVPAPITGTIIWFDPW,
(SEQ ID NO: 16)
CARGRSSSSYYMDVW,
(SEQ ID NO: 17)
CTHDWRNW,
(SEQ ID NO: 18)
CVKSAGRTSASPSYYYMDVW,
(SEQ ID NO: 19)
CARGRGRDCRSSNCYYMDVW,
and
(SEQ ID NO: 20)
CARGRSSSSYYMDVW.

FIG. 3 shows that the dominant antibody in TLS is an IgA

FIG. 4 shows kappa and lambda light chains for the TLS IgA antibodies

FIG. 5 shows the reactivity of the kappa light chain and lambda light chains.

FIGS. 6A and 6B show that the TLS derived antibody abrogates the growth of human ovarian cancer. FIG. 6A shows tumor volume, weight, and images of OVCAR3 tumors treated with IgA kappa antibody, IgA lambda antibody, irrelevant antibody, or vehicle controls. FIG. 6B shows tumor volume, weight, and images of primary cell line tumor treated with IgA kappa antibody, IgA lambda antibody, irrelevant antibody, or vehicle controls.

FIGS. 7A, 7B, 7C, and 7D show Glycosylated ACAT1 is the specific target of TLS-k antibody. FIG. 7A shows IP with TLS-derived antibody. FIG. 7B shows the binding of peptides of ACAT1 using Kappa light antibody or Lambda light chain antibody in 81908 primary cell line or OvCAR3 cells. FIG. 7C shows the intensity of staining for the conditions of 7B. FIG. 7D shows binding of glycosylated and deglycosylated primary cell lines and OVCAR3 cells by the TLS derived IgA kappa light chain antibody.

FIG. 8 shows that the most dominant TLS-derived antibody recognized only cancer cells' ACAT1 and that ovarian cancer cells secrete ACAT1 and Integrin β1 in high levels.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-ACAT1 binding molecule is disclosed and discussed and a number of modifications that can be made to a number of molecules including the anti-ACAT1 binding molecule are discussed, specifically contemplated is each and every combination and permutation of anti-ACAT1 binding molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are anti-anti-acetyl-CoA acetyltransferase 1 (ACAT1) binding molecules (such as, for example, antibodies, diabodies, nanobodies, scFv, sFv, and immunotoxins) that specifically binds glycosylated ACAT1, and wherein the anti-ACAT1 binding molecule comprising one or more variable heavy chain complementarity determining regions (CDRs) as set forth in SEQ ID Nos 1-3 (such as, for example the variable heavy chain as set forth in SEQ ID NO: 7) and one or more variable light chain complementarity determining regions (CDRs) as set forth in SEQ ID Nos 4-6 (such as, for example the variable light chain as set forth in SEQ ID NO: 9). In one aspect, the anti-ACAT1 binding molecule is an IgG, IgA, or IgM antibody.

### 1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with glycosylated ACAT1. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. For example, fragments of antibodies which maintain ACAT1 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M.J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti ACAT1 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject. along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K.D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### 4. Method of Treating Cancer

The disclosed anti-CAT1 binding molecules can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

In one aspect, the treatment of the cancer can include any of the ACAT1 binding molecules disclosed herein. Thus, disclosed herein are methods of treating, reducing, decreasing, inhibiting, ameliorating, and or preventing a cancer and/or metastasis (such as for example, ovarian, pancreatic, colorectal, bladder and non-small cell lung cancer) comprising administering to the subject any of the ACAT1 binding molecules disclosed herein. For example, disclosed herein are methods of treating, reducing, decreasing, inhibiting, ameliorating, and or preventing a cancer and/or metastasis (such as for example, ovarian, pancreatic, colorectal, bladder and non-small cell lung cancer) in a subject in need thereof comprising administering to the subject an anti-acetyl-CoA acetyltransferase 1 (ACAT1) binding molecule (such as, for example, antibodies, diabodies, nanobodies, scFv, sFv, and immunotoxins) that specifically binds glycosylated ACAT1, and wherein the anti-ACAT1 binding molecule comprising one or more variable heavy chain complementarity determining regions (CDRs) as set forth in SEQ ID Nos 1-3 (such as, for example the variable heavy chain as set forth in SEQ ID NO: 7) and one or more variable light chain complementarity determining regions (CDRs) as set forth in SEQ ID Nos 4-6 (such as, for example the variable light chain as set forth in SEQ ID NO: 9). In one aspect, the anti-ACAT1 binding molecule is an IgG, IgA, or IgM antibody.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel

Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumah, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP. Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica

(Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

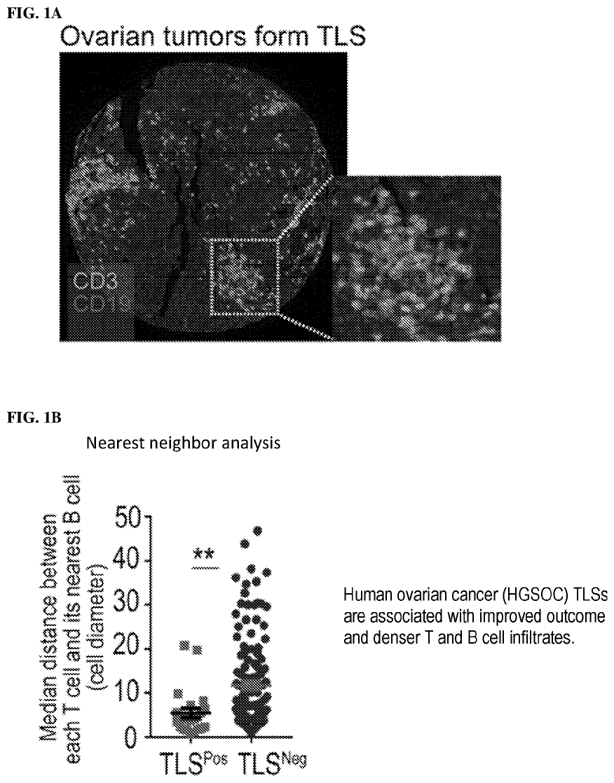

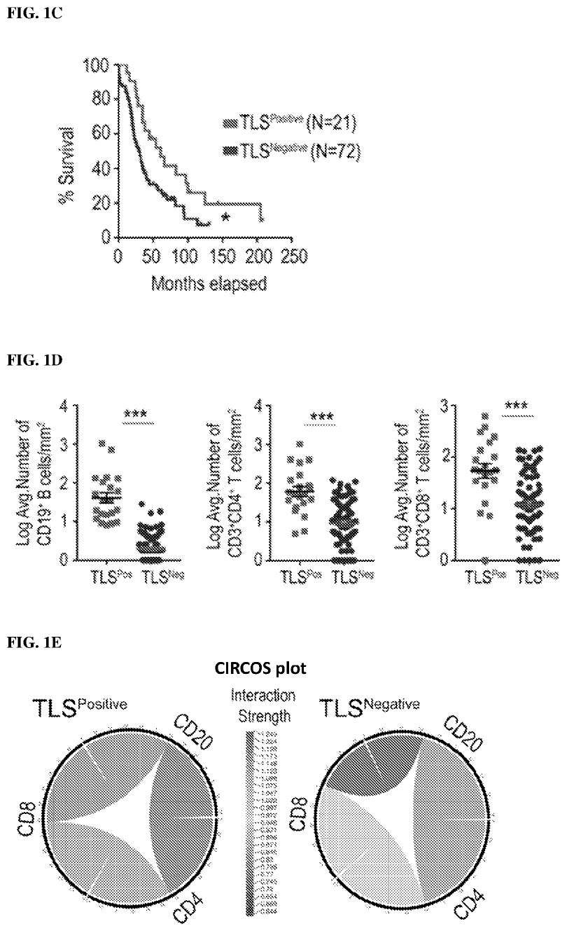

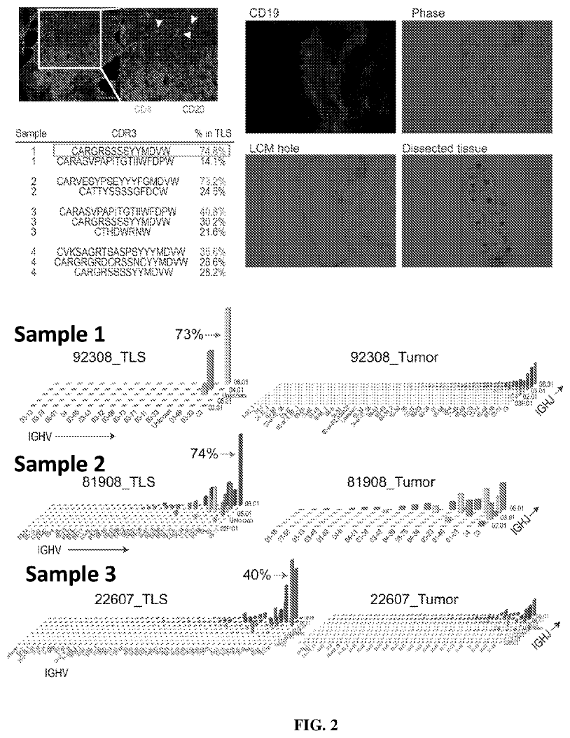

We wanted to examine the composition and density of B cell and T cells infiltrates in ovarian cancer to determine the difference between cancer with and without TLS. Using immunofluorescence we were able to characterize the CD3 positive cells (T cells) and CD19 positive cells (B cells) in ovarian cancer cells stained with DAPI (FIG. 1A). Using nearest neighbor analysis we observed that in TLS positive cancers a denser population of T and B cells (FIG. 1B). Looking at the survival of subjects with TLS compared to those without, we observed that Human ovarian cancer (HGSOC) tertiary lymphoid structures (TLSs) are associated with improved outcome (FIG. 1C). Looking at the number of B cells, CD4 T cells, and CD8T cells, we observed an increased number of B cells, CD4 T cells, and CD8T cells in TLS positive cancers compared to TLS negative cancers (FIG. 1D). Measuring the interaction strength and as shown by a CIRCOS plot, TLS positive cancers had a stronger interaction of B cells, CD4 T cells, and CD8T cells when compared with TLS negative cancers (FIG. 1E). We next examined the TLS from different human ovarian cancers (HGSOC) using laser microdissection (LCM) and adaptive immunosequencing (FIG. 2). Immunosequencing revealed that variable heavy chain (VH) sequences are highly clonal revealing 10 related sequences CARGRSSSSYYMDVW (SEQ ID NO: 11), CARASVPAPITGTIIWFDPW (SEQ ID NO: 12), CARVESYPSEYYYFGMDVW (SEQ ID NO: 13), CATTYSSSSGFDCW (SEQ ID NO:14), CARASVPAPITGTIIWFDPW (SEQ ID NO: 15), CARGRSSSSYYMDVW (SEQ ID NO: 16), CTHDWRNW (SEQ ID NO: 17), CVKSAGRTSASPSYYYMDVW (SEQ ID NO:18), CARGRGRDCRSSNCYYMDVW (SEQ ID NO: 19), and CARGRSSSSYYMDVW (SEQ ID NO: 20). Results showed a strong oligoclonal B cell response in the TLS areas, with a broader repertoire in whole tumor.

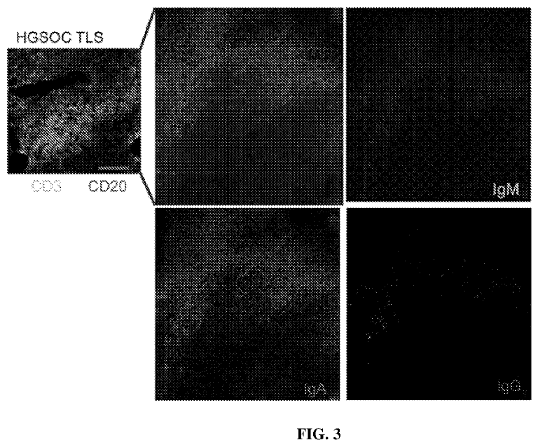

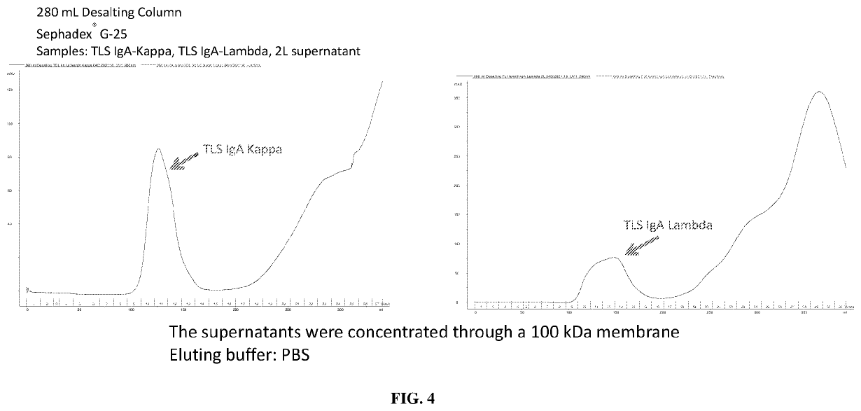

As shown in FIG. 2, the most dominant VH sequence (74.8% of clonotypes in that TLS). As shown in FIG. 3, we know that the most dominant antibody in TLS is an IgA. We then used multiplex PCR and sequencing to identify the two most dominant variable light chains (VL) sequences (one kappa and one lambda) (FIG. 4). We then generated dimeric IgA by combining the heavy chain in IgA backbone, with kappa and lambda light chains independently along with J-chain. To make these recombinant antibodies, the VH is cloned in PBMN-I GFP (retroviral transduction and GFP sort). Next the J-chain is cloned in pcDNA 3 (Lipofectamine transfection and G418/Geneticin selection). Finally, the light chain is cloned in pVITRO1 (Lipofectamine transfection and Hygromycin selection).

| VH chain (IGHV01-69/IGHD03-16/IGHJ06-01*02/IgA1): SP; | |
|---|---|
| CDR regions are underlined, Chothia prediction | |
| | M D W T W R F L F V V A A A T G V Q S Q |
| 1 | Atggactggacctggaggttcctctttgtggtggcagcagctacaggtgtccagtcccag |
| | V Q L V Q S G A E V K K P G S S V K V S |
| 61 | gtgcagctggtgcagtctggggctgaggtgaagaagcctgggtcctcggtgaaggtctcc |
| | C K A S G G T F S S Y A I S W V R Q A P |
| 121 | tgcaaggcttctggaggcaccttcagcagctatgctatcagctgggtgcgacaggecect |
| | G Q G L E W M G G I I P I F G T A N Y A |
| 181 | ggacaagggcttgagtggatgggagggatcatccctatctttggtacagcaaactacgca |
| | Q K F Q G R V T I T A D E S T S T A Y M |
| 241 | cagaagttccagggcagagtcacgattaccgcggaCGAATCCACGAGCACAGCCTACATG |
| | E L T G L T S Q D T A V Y Y C A R E D I |
| 301 | GAGCTGACCGGCCTGACATCTCAAGACACGGCCGTATACTACTGCGCGAGAGAAGACATA |
| | L M I Y S Y F G M D V W G Q G T T V T V |
| 361 | CTTATGATTTATTCGTACTTCGGCATGGACGTCTGGGGCCAAGGGaccacggtcaccgtc |
| | S S |
| 421 | tcctcag |
| VL chain: SP; CDR regions are underlined, Chothia predicitons) | |
| | M D W T W R I L F L V A A A T G A H S E |
| 1 | ATGGACTGGACCTGGAGGATCCTCTTCTTGGTGGCGGCCGCCACAGGCGCGCACTCCGAG |
| | V Q L V E S G G G L V Q P G G S L R L S |
| 61 | GTGCAGCTGGTGGAGTCTGGTGGTGGTCTTGTTCAACCTGGTGGTTCTCTTCGTCTTTCT |
| | C A A S G F N I K D T Y I H W V R Q A P |
| 121 | TGTGCTGCTTCTGGTTTTAATATTAAAGATACTTATATTCATTGGGTTCGTCAAGCTCCT |
| | G K G L E W V A R I Y P T N G Y T R Y A |
| 181 | GGTAAAGGTCTTGAATGGGTTGCTCGTATTTATCCTACTAATGGTTATACTCGTTATGCT |
| | D S V K G R F T I S A D T S K N T A Y L |
| 241 | GATTCTGTTAAAGGTCGTTTTACTATTTCTGCTGATACTTCTAAAAATACTGCTTATCTT |
| | Q M N S L R A E D T A V Y Y C S R W G G |
| 301 | CAAATGAACTCTCTTCGTGCTGAAGATACTGCTGTTTATTATTGTTCTCGTTGGGGTGGT |
| | D G F Y A M D Y W G Q G T L V T |
| 361 | GATGGTTTTTATGCTATGGATTATTGGGGTCAAGGTACTCTTGTCAC 407 |

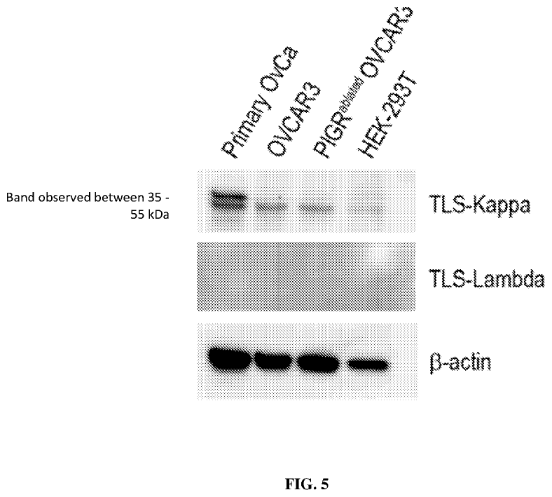

Next, we examined the reactivity of the kappa and lambda antibodies. The recombinant antibody with the kappa light chain sequence was able to identity a target in tumor lysates and other tumors. By contrast, the antibody with a lambda light chain did not bind any targets (FIG. 5).

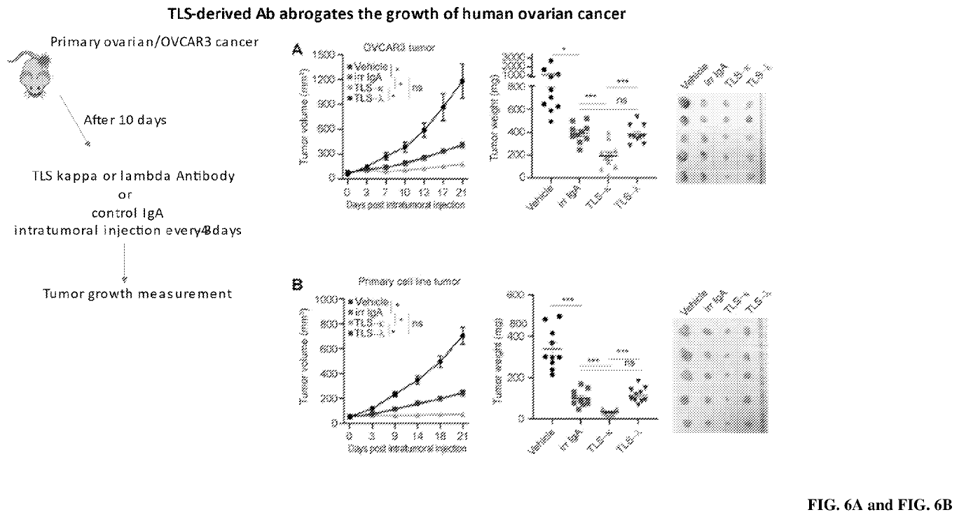

To determine the effect of the TLS-derived antibodies on cancer cells, we implanted OVCAR3 cancer cells or primary cancer cells from an ovarian tumor in mice. After 10 days, Recombinant antibodies comprising a Kappa light chain (TLS-κ), Lambda light chain (TLS-λ), or a control IgA were administered via intratumoral injection every 3-4 days and tumor growth was measured. As shown in FIG. 6, TLS-κ derived antibody abrogates the growth of human ovarian cancer. Tumor volume, weight, and images of OVCAR3 (FIG. 6A) and primary cancer 81908 (FIG. 6B) tumors treated with IgA kappa antibody, IgA lambda antibody, irrelevant antibody, or vehicle controls. While the TLS-λ antibody showed no improvement over an irrelevant antibody, TLS-κ antibodies showed marked improvement in reducing tumor volume and weight in both cancers.

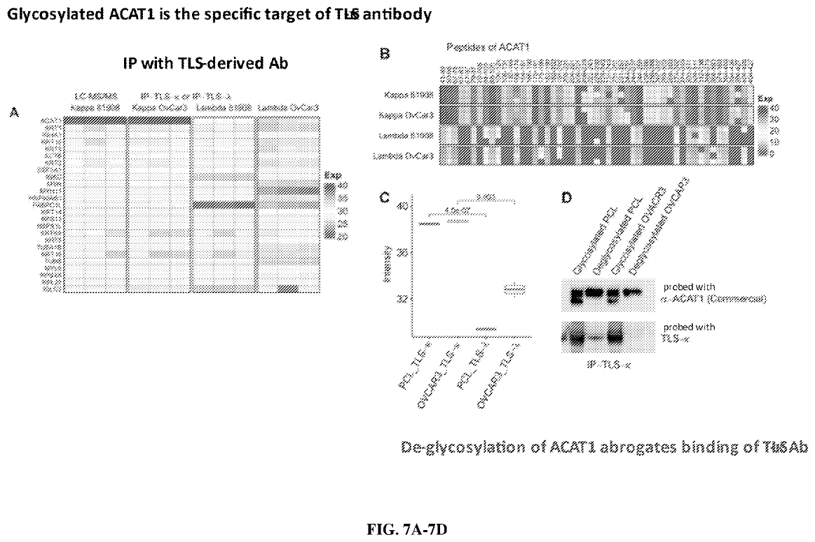

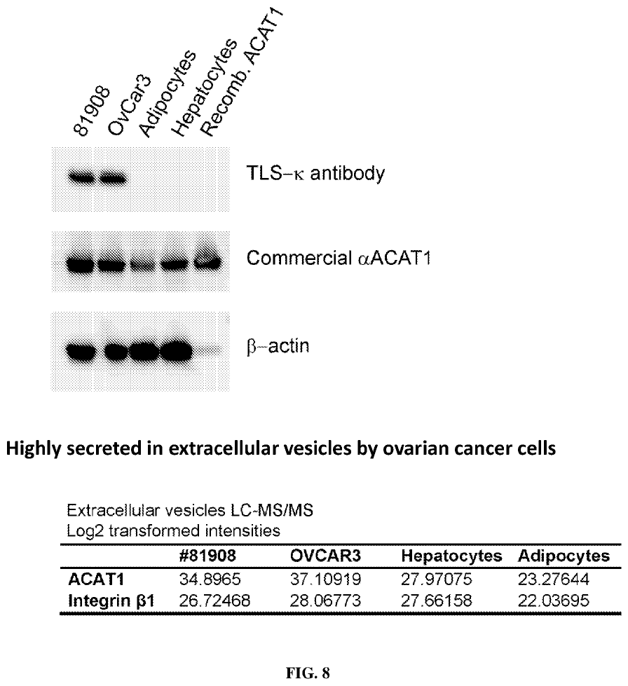

We then assayed the binding of the TLS-κ and TLS-λ antibodies to determine their target. As shown in FIG. 7A, the TLS-k antibodies target ACAT1, wherein the TLS-λ antibodies had no specific target. We then measured the antibodies against peptides of ACAT1 to identify specific peptide targets (FIG. 7B) and binding intensity (FIG. 7C). To see if the targets were glycosylated or not, we compared the binding to TLS-κ in both OVCAR3 and primary cancer cells (PCL) that had been glycosylated or deglycosylated. Staining showed that TLS-K strongly associated with glycosylated targets on at best only weakly associated with deglycosylated targets (FIG. 7D). Checking the specificity of the TLS-k antibody we observed binding only in cancers with ACAT1 FIG. 8).

### D. Sequences

SEQ ID NO: 1
VH chain CDR1
GGTFSSY
SEQ ID NO: 2
VH chain CDR2
IPIFG
SEQ ID NO: 3
VH chain CDR3
EDILMIYSYFGMD
SEQ ID NO: 4
VL chain CDR1
GFNIKDT
SEQ ID NO: 5
VL chain CDR2
YPTNGY
SEQ ID NO: 6
VL chain CDR3
SRWGGDG
SEQ ID NO: 7:
anti-ACAT1 variable heavy chain amino acid
sequence
MDWTWRFLFVVAAATGVQSQVQLVQSGAEVKKPGSSVKVSCKASG
GTFSSYATSWVRQAPGQGLEWMGGTTPTFGTANYAQKFQGRVTTT
ADESTSTAYMELTGLTSQDTAVYYCAREDTLMTYSYFGMDVWGQG
TTVTVSS
SEQ ID NO: 8:
anti-ACAT1 variable heavy chain nucleic acid
sequence
ATGGACTGGACCTGGAGGTTCCTCTTTGTGGTGGCAGCAGCTACA
GGTGTCCAGTCCCAGGTGCAGCTGGTGCAGTCTGGGGCTGAGGTG
AAGAAGCCTGGGTCCTCGGTGAAGGTCTCCTGCAAGGCTTCTGGA
GGCACCTTCAGCAGCTATGCTATCAGCTGGGTGCGACAGGCCCCT
GGACAAGGGCTTGAGTGGATGGGAGGGATCATCCCTATCTTTGGT
ACAGCAAACTACGCACAGAAGTTCCAGGGCAGAGTCACGATTACC
GCGGACGAATCCACGAGCACAGCCTACATGGAGCTGACCGGCCTG
ACATCTCAAGACACGGCCGTATACTACTGCGCGAGAGAAGACATA
CTTATGATTTATTCGTACTTCGGCATGGACGTCTGGGGCCAAGGG
ACCACGGTCACCGTCTCCTCAG
SEQ ID NO: 9:
anti-ACAT1 variable light chain amino acid
sequence
MDWTWRTLFLVAAATGAHSEVQLVESGGGLVQPGGSLRLSCAASG
FNTKDTYTHWVRQAPGKGLEWVARTYPTNGYTRYADSVKGRFTTS
ADTSKNTAYLQMNSLRAEDTAVYYCSRWGGDGFYAMDYWGQGTLV
T
SEQ ID NO: 10:
anti-ACAT1 variable light chain nucleic acid
sequence
ATGGACTGGACCTGGAGGATCCTCTTCTTGGTGGCGGCCGCCACA
GGCGCGCACTCCGAGGTGCAGCTGGTGGAGTCTGGTGGTGGTCTT
GTTCAACCTGGTGGTTCTCTTCGTCTTTCTTGTGCTGCTTCTGGT
TTTAATATTAAAGATACTTATATTCATTGGGTTCGTCAAGCTCCT
GGTAAAGGTCTTGAATGGGTTGCTCGTATTTATCCTACTAATGGT
TATACTCGTTATGCTGATTCTGTTAAAGGTCGTTTTACTATTTCT
GCTGATACTTCTAAAAATACTGCTTATCTTCAAATGAACTCTCTT
CGTGCTGAAGATACTGCTGTTTATTATTGTTCTCGTTGGGGTGGT
GATGGTTTTTATGCTATGGATTATTGGGGTCAAGGTACTCTTGTC
AC
SEQ ID NO: 11
Variable Heavy Chain CDR3 amino acid sequence
CARGRSSSSYYMDVW
SEQ ID NO: 12
Variable Heavy Chain CDR3 amino acid sequence
CARASVPAPITGTIIWFDPW
SEQ ID NO: 13
Variable Heavy Chain CDR3 amino acid sequence
CARVESYPSEYYYFGMDVW
SEQ ID NO:14
Variable Heavy Chain CDR3 amino acid sequence
CATTYSSSSGFDCW
SEQ ID NO: 15
Variable Heavy Chain CDR3 amino acid sequence
CARASVPAPITGTIIWFDPW
SEQ ID NO: 16
Variable Heavy Chain CDR3 amino acid sequence
CARGRSSSSYYMDVW
SEQ ID NO: 17
Variable Heavy Chain CDR3 amino acid sequence
CTHDWRNW
SEQ ID NO: 18
Variable Heavy Chain CDR3 amino acid sequence
CVKSAGRTSASPSYYYMDVW
SEQ ID NO: 19
Variable Heavy Chain CDR3 amino acid sequence
CARGRGRDCRSSNCYYMDVW
SEQ ID NO: 20
Variable Heavy Chain CDR3 amino acid sequence
CARGRSSSSYYMDVW

1. An anti-acetyl-CoA acetyltransferase 1 (ACAT1) binding molecule that specifically binds glycosylated ACAT1, and wherein the anti-ACAT1 binding molecule comprising one or more variable heavy chain complementarity determining regions (CDRs) as set forth in SEQ ID Nos 1-3 and one or more variable light chain complementarity determining regions (CDRs) as set forth in SEQ ID Nos 4-6.

2. The anti-ACAT1 binding molecule of claim 1, wherein the variable heavy chain comprises the sequence as set forth in SEQ ID NO: 7.

3. The anti-ACAT1 binding molecule of claim 1, wherein the variable light chain comprises the sequence as set forth in SEQ ID NO: 9.

4. The anti-ACAT1 binding molecule of claim 1, wherein the anti-ACAT1 binding molecule is an IgG, IgA, or IgM antibody.

5. A method of treating a cancer in a subject comprising administering to the subject the ACAT1 binding molecule of claim 1.

6. A method of treating a cancer in a subject in need thereof comprising administering to the subject an anti-acetyl-CoA acetyltransferase 1 (ACAT1) binding molecule that specifically binds glycosylated ACAT1, and wherein the anti-ACAT1 binding molecule comprising one or more variable heavy chain complementarity determining regions (CDRs) as set forth in SEQ ID Nos 1-3 and one or more variable light chain complementarity determining regions (CDRs) as set forth in SEQ ID Nos 4-6.

7. The method of treating a cancer of claim 5, wherein the cancer comprises ovarian cancer.

8. The method of treating a cancer of claim 6, wherein the variable heavy chain comprises the sequence as set forth in SEQ ID NO: 7.

9. The method of treating a cancer of claim 6, wherein the variable light chain comprises the sequence as set forth in SEQ ID NO: 9.

10. The method of treating a cancer of claim 6, wherein the anti-ACAT1 binding molecule is an IgG, IgA, or IgM antibody.

11. The method of treating a cancer of claim 6, wherein the cancer comprises ovarian cancer.
